(19) United States
(12) Patent Application Publication
(10) Pub. No.: US 2005/0131071 A1
(43) Pub. Date: Jun. 16, 2005

(54) Orodispersible pharmaceutical composition of agomelatine

(76) Inventors: Patrick Wuthrich, Orleans (FR); Herve Rolland, Olivet (FR); Marc Julien, Sigloy (FR); Francois Tharrault, Orleans (FR)

Correspondence Address: THE FIRM OF HUESCHEN AND SAGE, 500 COLUMBIA PLAZA, 350 EAST MICHIGAN AVENUE, KALAMAZOO, MI, 49007, US

(21) Appl. No.: 10/502,593

(22) PCT Filed: Jan. 22, 2003
(86) PCT No.: PCT/FR03/00197

(30) Foreign Application Priority Data
Jan. 23, 2003 (FR) 0200792

(51) Int. Cl. A61K 31/16; A61K 9/20

(52) U.S. Cl. 514/630; 424/464

(57) ABSTRACT

The invention relates to a solid orodispersible pharmaceutical composition of agomelatine, characterised in that it comprises agomelatine and granules consisting of co-dried lactose and starch.

The present invention relates to a solid orodispersible pharmaceutical form for the administration of agomelatine by the oral route.

Agomelatine, or N-[2-(7-methoxy-1-naphthyl)ethyl]acetamide, is a selective agonist of melatoninergic receptors.

Agomelatine can be administered by the oral route in the form of immediate-release tablets to be swallowed with half a glass of water. Those agomelatine tablets are of use especially in the treatment of depression, sleep disorders and all pathologies associated with deregulation of circadian rhythms.

Pharmacokinetic studies in humans have shown that the bioavailability of agomelatine by the oral route is very low in relation to the parenteral route and is subject to considerable variation within one and the same individual and from one individual to another.

The low bioavailability of agomelatine and the variations in inter- and intra-individual concentrations have therefore resulted in the search for a new formulation allowing those problems to be solved.

The pharmaceutical compositions of the present invention make it possible not only to solve the known problems of the immediate-release oral form but also to offer a superior medical service which especially allows the quality of life of patients to be improved.

The orodispersible pharmaceutical composition of agomelatine has the advantage that elevated plasma levels of active ingredient are obtained rapidly whilst avoiding the significant metabolisation of the active ingredient due to the hepatic first-pass effect.

The orodispersible pharmaceutical composition according to the invention has the particular characteristic of requiring neither water nor chewing in the course of its administration. It disintegrates very rapidly in the mouth, preferably in less than three minutes and even more preferably in less than one minute. It is administered, preferably but not exclusively, under the tongue.

Many rapid-dissolution forms are described in the prior art. In general, it is common to the previously described technologies that they use a disintegrating agent such as Kollidon® CL (crosslinked polyvinylpyrrolidone), EXPLOTAB® (carboxymethyl starch) and AC DISOL® (crosslinked sodium carboxymethylcellulose).

That disintegrating agent is indispensable to the formulation of the orodispersible tablets and has to be used in conjunction with a direct-compression excipient. The difficulties encountered in the manufacture of such tablets reside in the fact that it is very difficult to obtain tablets having physical characteristics that are constant and reproducible and compatible with the customary handling requirements of tablets.

However, the customarily used mixtures result in tablets of very considerable hardness which is completely unsuitable for rapid disintegration in the oral cavity.

Other orodispersible forms can be produced by using lyophilisation, resulting in very porous solid forms called “oral lyophilisates”. Those forms require the use of a highly specific and complicated industrial process which is lengthy to carry out, yielding a medicament form which has a high cost price.

The present invention enables those problems to be solved. It relates to a solid orodispersible form of agomelatine comprising a single excipient of natural origin which allows rapid disintegration and which has a neutral flavour and agreeable texture. The said excipient acts both as binder and as disintegrant. It allows a simple agomelatine formulation to be obtained, having excellent suitability for direct compression, resulting in tablets of low friability and of a hardness that is compatible with customary handling methods.

More specifically, the invention relates to a solid orodispersible pharmaceutical composition of agomelatine, characterised in that it comprises:
agomelatine,
and granules consisting of co-dried lactose and starch.

The composition according to the invention may also comprise, for reasons of manufacture, one or more lubricants and a flow agent, as well as flavourings, colourings and sweetening agents as conventionally used.

In order to improve local tolerance of the agomelatine (reduction in the sensation of tingling), the agomelatine may optionally be associated with excipients such as cyclodextrins or coated with excipients using technologies known to the person skilled in the art such as, for example, coating in a fluidised-air bed, atomisation and coacervation.

The invention relates also to the use of granules consisting of co-dried lactose and starch in the manufacture of solid orodispersible pharmaceutical compositions of agomelatine.

The term “orodispersible” is understood to refer to solid pharmaceutical compositions which disintegrate in the oral cavity in less than 3 minutes, preferably less than one minute.

The said granules present in the solid pharmaceutical compositions according to the invention correspond to the compositions described in Patent Application EP 00/402159.8. Those granules are characterised by a spherical structure and an advantageous compressibility and are marketed under the name STARLAC®.

The disintegrating properties of the said granules are known for tablets placed in large volumes of stirred liquids. It is especially surprising that, when used in the manufacture of orodispersible forms, the said granules should give especially satisfactory results in terms of disintegration in the mouth, for two reasons.

The first reason is based on the finding that the least water-soluble excipients are the most suitable for the formulation of orodispersible tablets (dissolution, in bringing about an increase in the viscosity of water, slows down its penetration into the tablets) and yet the said granules contain a large amount of highly water-soluble lactose. Moreover, the starch contained in the said granules is not a “super-disintegrant” agent as used and described in the orodispersible forms of the prior art.

The second is based on the finding that the disintegrant properties of an excipient (used in a tablet), when determined in water using conventional methods, cannot be extrapolated to the behaviour of the same tablet in vivo, in saliva. Disintegration rates in water are measured (in accordance with the European Pharmacopoeia) in an amount of water that is sufficiently large not to reach saturation level in terms of dissolution, whereas in vivo, by virtue of the small volume of saliva, the excipients are at saturation level. Furthermore, the stirring to which the tablets are subjected in the customary test does not reflect disintegration in the mouth. The Applicant accordingly found, during comparative tests, that certain excipients which are known as good disintegrants are not suitable for the preparation of orodispersible forms. Conversely, certain excipients that exhibit average disintegration in water may exhibit advantageous properties in vivo.

The Applicant then found, surprisingly, that the said granules rendered the tablets highly suitable for disintegration in the mouth, that being the case over a wide tablet hardness range, whilst maintaining a low level of friability, which is especially remarkable. Most orodispersible forms of the prior art which disintegrate rapidly in the mouth are highly friable, which is reflected by the need to use a specific packaging and the risk of the tablet disintegrating as soon as it is handled and taken out of its pack.

It is especially remarkable that the above-mentioned criteria of orodispersibility and low friability are maintained over a wide tablet hardness range, that is to say for tablets having a hardness of from 15 to 30 Newtons.

The pharmaceutical compositions according to the invention are preferably characterised in that they comprise, in relation to the total weight of the tablet:
from 0.2% to 10% by weight of agomelatine,
from 85% to 98.5% by weight of STARLAC®.

They may optionally comprise from 0.1% to 3% by weight of lubricating agents such as magnesium stearate or sodium stearyl fumarate, preferably from 0.5% to 1.5%, and from 0.1% to 3% by weight of a flow agent such as colloidal silica, preferably from 0.5% to 1.5%.

The following Examples illustrate the invention without limiting it in any way:

Orodispersible agomelatine tablets

### EXAMPLE 1

### Formulation: Finished tablet of 50 mg

| Constituents | Amount (mg) |
|---|---|
| Agomelatine | 0.5 |
| Starlac ® | 48.5 |
| Magnesium stearate | 0.5 |
| Anhydrous colloidal silica | 0.5 |

### EXAMPLE 2

### Formulation: Finished tablet of 100 mg

| Constituents | Amount (mg) |
|---|---|
| Agomelatine | 5 |
| Starlac ® | 93.5 |
| Magnesium stearate | 1 |
| Anhydrous colloidal silica | 0.5 |

The tablets are prepared by mixing the constituents, followed by direct compression. The hardness of the tablets of Examples 1 and 2 is about 20 Newtons.

In order to determine the disintegration time in the mouth, the orodispersible agomelatine tablets described in Examples 1 and 2 were placed under the tongue in order to promote the systemic passage of agomelatine by the sublingual route and to avoid as far as possible the hepatic first-pass effect.

In these tests it was found that, for each of the formulations tested, the disintegration time in the mouth was less than 1 minute.

1-9. (canceled)

10. A solid orodispersible pharmaceutical composition comprising:
granules consisting of co-dried lactose and starch and
agomelatine.

11. A composition according to claim 10 wherein the composition disintegrates in the mouth in less than three minutes.

12. A composition according to claim 11 wherein the composition disintegrates in the mouth in less than one minute.

13. A composition according to claim 10, comprising, in relation to the total weight of the composition:
from 85% to 98.5% by weight of granules consisting of co-dried lactose and starch and
from 0.2% to 10% by weight of agomelatine.

14. A composition according to claim 10, further comprising one or more lubricants and a flow agent.

15. A composition according to claim 10, wherein the composition is in the form of a tablet.

16. A tablet according to claim 15, wherein the tablet is obtained by direct compression.

17. A tablet according to claim 16, wherein the tablet has a hardness from 15 to 50 Newtons.

18. A tablet according to claim 17, wherein the tablet has a hardness of about 20 Newtons.

19. A process for the manufacture of solid orodispersible compositions of agomelatine which disintegrate in the mouth in less than three minutes, wherein the agomelatine is mixed with granules consisting of co-dried lactose and starch.

20. A process for the manufacture of solid orodispersible compositions of agomelatine which disintegrate in the mouth in less than one minute, wherein the agomelatine is mixed with granules consisting of co-dried lactose and starch.

21. A method for treating a living animal body, including a human, afflicted with a condition selected from depression, sleep disorders and pathologies associated with deregulation of circadian rhythms, comprising the step of administering to the living animal body, including a human, a composition according to claim 10 which is effective for alleviation of the condition.
